(19) United States
(12) Patent Application Publication
(10) Pub. No.: US 2015/0245787 A1
(43) Pub. Date: Sep. 3, 2015

(54) REAL-TIME VIDEO PROCESSING FOR RESPIRATORY FUNCTION ANALYSIS

(71) Applicant: Xerox Corporation, Norwalk, CT (US)

(72) Inventors: Survi KYAL, Rochester, NY (US); Lalit Keshav MESTHA, Fairport, NY (US); Himanshu J. MADHU, Webster, NY (US)

(73) Assignee: XEROX CORPORATION, Norwalk, CT (US)

(21) Appl. No.: 14/195,111

(22) Filed: Mar. 3, 2014

(51) Int. Cl. A61B 5/08; H04N 5/357; A61B 5/00; H04N 7/18

(52) U.S. Cl. CPC A61B 5/0816; H04N 7/183; H04N 5/357; A61B 5/0826; A61B 5/4818

(57) ABSTRACT

What is disclosed is a system and method for processing a video for respiratory function analysis. In one embodiment, a video is received of a region of the subject's body where a time-varying signal corresponding to the subject's respiration can be registered by the video camera. Pixels in a first batch of frames are processed to obtain a time-series signal which is filtered using a band-pass filter with a low and high cutoff frequency f_{L} and f_{H}, where f_{L} and f_{H} are a function of the subject's tidal breathing. The filtered time-series signal is analyzed to identify a next low and high cutoff frequency f′_{L} and F′_{H}, where f_{L}<f′_{L} and f′_{H}<f_{H}. Thereafter, next successive batch of frames are repeatedly processed to obtain respective next time-series signal which is filtered using a band-pass filter with the cutoff frequency f′_{L} and f′_{H}. The filtered signals are processed to obtain a respiratory signal.

### TECHNICAL FIELD

The present invention is directed to systems and methods for real-time processing of a video of a subject for respiratory function analysis in a non-contact, remote sensing environment.

### BACKGROUND

Monitoring of patient respiratory events is of vital clinical importance in the early detection of potentially fatal conditions. Current technologies that involve contact sensors require that the individual wears such devices constantly. Such a requirement can lead to discomfort, psychological dependence, loss of dignity, and may even cause additional medical issues such as skin infection when sensors have to be worn for an extended period of time. Elderly patients, infants, and those suffering from chronic medical conditions are more likely to suffer from such negative effects of continuous monitoring. The use of an unobtrusive, non-contact, imaging based monitoring of respiratory events can go a long way towards alleviating some of these issues. Continuous monitoring of patient respiration rate in a non-contact, remote sensing environment is of vital clinical importance.

Accordingly, what is needed in this art are increasingly sophisticated systems and methods for real-time processing of a video of a subject for respiratory function analysis in a non-contact, remote sensing environment.

### INCORPORATED REFERENCE

The following U.S. patents, U.S. Patent Applications, and Publications are incorporated herein in their entirety by reference.

“Monitoring Respiration with a Thermal Imaging System”, U.S. patent application Ser. No. 13/103,406, by Xu et al.

Using An Adaptive Band-Pass Filter To Compensate For Motion Induced Artifacts In A Physiological Signal Extracted From Video”, U.S. patent application Ser. No. 14/099,358, Kyal et al.

### BRIEF SUMMARY

What is disclosed is a system and method for real-time processing of a video of a subject for respiratory function analysis in a non-contact, remote sensing environment. In one embodiment, the present method involves the following. First, a video is received. The video comprises a plurality of time-sequential image frames of a subject being monitored for respiratory function. The video images can be any combination of RGB images, IR images, multispectral images, and hyperspectral video images. The video is of a region of the subject's body where a time-varying signal corresponding to the subject's respiration can be registered by the video camera. Then, pixels in a first batch of image frames are processed to obtain a first time-series signal. This first time-series signal is filtered using a band-pass filter having a low cutoff frequency f_{L} and a high cutoff frequency f_{H}>f_{L}, where f_{L} and f_{H} are at least a function of the subject's tidal breathing. The filtered first time-series signal is analyzed to identify a next low cutoff frequency f′_{L} and a next high cutoff frequency f′_{H}, where f_{L}<f′_{L}<f′_{H}<f_{H}. Thereafter, the following steps are performed for each next successive batch of image frames. Pixels in the next successive batch of image frames are processed to obtain a next sequential time-series signal. The next time-series signal is filtered using a band-pass filter with the identified next low and high cutoff frequencies. The filtered time-series signal is processed to obtain a respiratory signal for the subject for this batch of image frames.

Features and advantages of the above-described method will become readily apparent from the following detailed description and accompanying drawings.

### BRIEF DESCRIPTION OF THE DRAWINGS

The foregoing and other features and advantages of the subject matter disclosed herein will be made apparent from the following detailed description taken in conjunction with the accompanying drawings, in which:

FIG. 1 is a flow chart which illustrates one example embodiment of the present method for processing image frames of a video of a subject for respiratory function analysis;

FIG. 2 is a continuation of the flow diagram of FIG. 1 with flow processing continuing with respect to node A; and

FIG. 3 shows a block diagram of one example video processing system 300 for processing a video in accordance with the embodiment shown and described with respect to the flow diagrams of FIGS. 1-2.

### DETAILED DESCRIPTION

What is disclosed is a system and method for processing image frames of a video of a subject for respiratory function analysis.

### Non-Limiting Definitions

A “respiratory function” refers to a function of the respiratory system of a subject.

A “subject” is a living person with a respiratory system. Although the term “person” or “patient” may be used throughout this text, it should be appreciated that the subject may be something other than a human. Such terms are not to be viewed as limiting the scope of the appended claims strictly to human beings. A video of the subject is received for processing in accordance with the methods disclosed herein.

A “video”, as is generally understood, is a plurality of time-sequential image frames of a body region of a subject where a respiratory signal corresponding to the subject's respiratory function can be registered by the video imaging device used to capture the video. The received video may be any combination of: monochrome images, color images, infrared (IR) images, multispectral, and hyperspectral images. The video may be processed or pre-processed to compensate for non-uniform illumination due to a curvature of a surface of the skin, for motion induced blur due to body or surface motion, imaging blur, and slow illuminant variation. Motion in the video may be compensated for using, for example, a video-based 2D or 3D surface stabilization techniques. The video may also contain other components such as, audio, time, frame rate data, and the like. The video of the subject is received for processing.

“Receiving a video” is intended to be widely construed and includes: retrieving, receiving, capturing, acquiring, or otherwise obtaining video for processing. For instance, image frames comprising the video can be retrieved from a memory or storage device of the video imaging device, or obtained from a remote device over a network. Video image frames may be retrieved from a media such as a CDROM or DVD, or downloaded from a web-based application which makes batches of video image frames available for processing. The received video was captured by a video imaging device.

A “video imaging device” is a single-channel or a multi-channel video camera which captures time-sequential image frames of a body region of the subject. The video imaging device may have a high frame rate and high spatial resolution such as, for example, a monochrome video camera, a RGB video camera, or a video capture device with thermal, infrared, multi-spectral or hyperspectral sensors. The video imaging device may comprise a hybrid device capable of operating in a conventional video mode with high frame rates and high spatial resolution, and a spectral mode with low frame rates but high spectral resolution. Various forms of structured and/or unstructured illumination may be utilized in conjunction with the video imaging device. Video imaging devices with standard imaging sensors and those with specialized sensors are available in various streams of commerce.

A “body region” refers to an area of the subject's body which contains at least a partial view of the subject's thoracic region (chest area) where a respiratory signal corresponding to the subject' respiratory function can be registered by a video imaging device. Body regions where a respiratory signal can be registered include the subject's anterior thoracic region, a side view of the thoracic region, and a back region of the dorsal body. Preferably, the body region is an area of exposed skin. Pixels are isolated in the image frames of the body region using image processing techniques such as pixel classification, object identification, thoracic region recognition, color, texture, spatial features, spectral information, pattern recognition, and a user input.

A “batch of video image frames” refers to a plurality of time-sequential image frames of the received video. In accordance with the teachings hereof, pixels associated with a body region of the subject wherein a respiratory signal is registered by the video imaging device are isolated in the image frames and the isolated pixels processed to obtain a time-series signal. Batches of image frames do not have to be the same size and may vary dynamically during processing. A size of a given batch of image frames can be pre-defined by the user depending on the application wherein the teachings hereof find their intended uses. A size of a batch of video image frames should be at least 3 breathing cycles of the subject. In one embodiment, batches of video frames are created by sliding a window of length 30 (or 15) seconds with 96.67% overlap between consecutive batches which means using only 1 second of new frames and retaining 29 seconds of frames from the previous batch. Processing a batch of video image frames generates a time-series signal on a per batch basis.

A “time-series signal” is a signal extracted from a batch of video image frames. The time-series signal contains frequency components related to the motion of the chest cage due to respiration. A respiration signal can be extracted from the time-series signal. A time series signal is generated by processing pixels in respective batches of video image frames in one or more areas of the subject's chest region. One such method, for example, averages pixel values within an isolated areas across a plurality of image frames. An average is computed of all pixels in each of the isolated areas within each image frame to obtain a channel average per frame. A global channel average is computed, for each channel, by adding the channel averages across multiple frames and dividing by the total number of frames. The channel average is subtracted from the global channel average and the result is divided by a global channel standard deviation to obtain a zero-mean unit variance time-series signal for that video segment. The time-series signal contains frequency components. The time-series signals may be normalized and are then subjected to a pre-filtering to remove undesirable frequencies. Various time-series signal segments can be weighted, as desired. Such a weighting may be applied over one or more segments while other signal segments are not weighted. Methods for weighting signal segments are widely understood in the signal processing arts. It should be appreciated that the time-series signal may be received or retrieved from a remote device such as a computer workstation over a wired or wireless network with the captured video having been communicated directly to the remote device for generation of the time-series signal on a continuous basis. Time-series signals extracted from the video are processed to extract a respiratory signal.

A “respiratory signal” is a signal obtained from having processed video of the subject and is used for respiratory function analysis and assessment. The respiratory signal can be obtained from the time-series signal by performing a non-parametric spectral density estimation on the filtered time-series signal, by performing a parametric spectral density estimation on the filtered time-series signal, and by performing automatic peak detection on the filtered time-series signal. The obtained respiratory signal can be analyzed to determine breathing pattern, and respiration rate. These can, in turn, be used to determine a condition related to any of: Sudden Infant Death Syndrome, respiratory distress, respiratory failure, apnea, and pulmonary disease.

A “band-pass filter” is used herein to filter the time-series signal. The band-pass filter has a low cutoff frequency f_{L} and a high cutoff frequency f_{H}, where f_{L} and f_{H} are a function of the subject's tidal breathing rate. The low and high cutoff frequencies can also be a function of the subject's respiratory health and age. As is generally understood with respect to band-pass filter, the filter's cut-off frequencies are selected so that the filter retains desirable components while rejecting undesirable components.

### Flow Diagram of One Example Embodiment

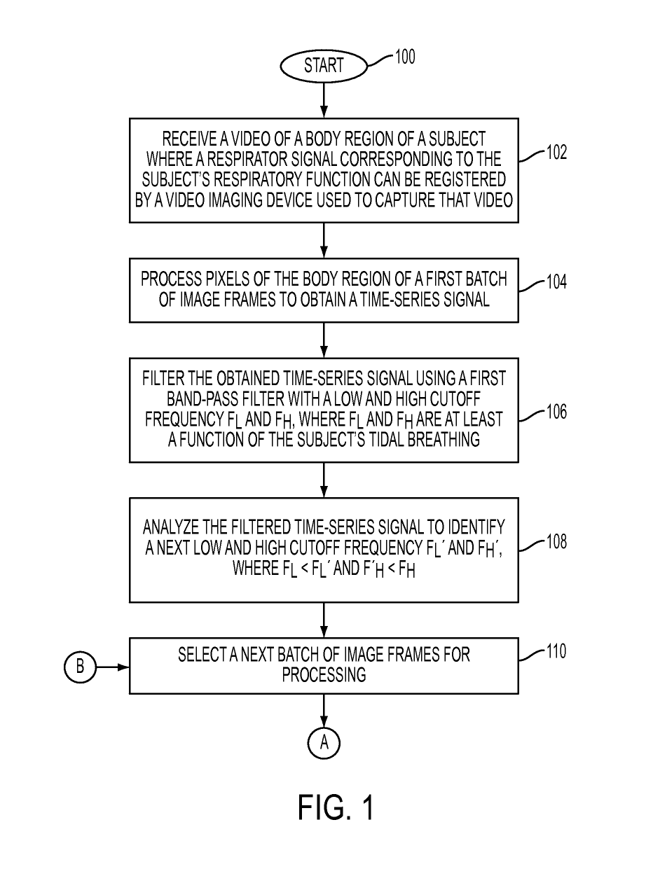

Reference is now being made to the flow diagram of FIG. 1 which illustrates one example embodiment of the present method for processing image frames of a video of a subject for respiratory function analysis. Flow processing begins at step 100 and immediately proceeds to step 102.

At step 102, receive a video of a body region of a subject where a time-varying signal corresponding to a respiratory function is registered by a video camera.

At step 104, process pixels in the body region of a first batch of image frames to obtain a time-series signal.

At step 106, filter the time-series signal using a first band-pass filter with a low and high cutoff frequency f_{L} and f_{H}, where f_{L} and f_{H} are at least a function of the subject's tidal breathing.

At step 108, analyze the filtered time-series signal to identify a low and high cutoff frequency f′_{L} and f′_{H}, where f_{L} <f′_{L} and f′_{H}<f_{H}.

At step 110, select a next batch of image frames for processing.

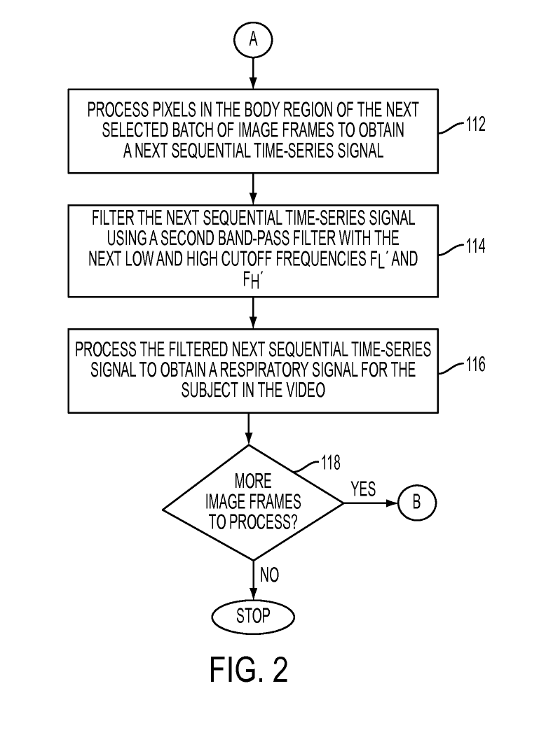

Reference is now being made to FIG. 2 which is a continuation of the flow diagram of FIG. 1 with flow processing continuing with respect to node A.

At step 112, process pixels in the body region of the next selected batch of image frames to obtain a next sequential time-series signal.

At step 114, filter the next sequential time-series signal using a second band-pass filter with the low and high cutoff frequencies f′_{L} and f′_{H} (identified in step 108). In one embodiment, the first and second band-pass filters are the same band-pass filter which is capable of having its low and high cutoff frequencies dynamically modified.

At step 116, process the filtered next sequential time-series signal to obtain a respiratory signal for the subject in the video. The generated respiratory signal is communicated to a display device.

At step 118, a determination is made whether more batches of video image frames remain to be processed. If so then processing repeats with respect to node B wherein, at step 110, a next batch of image frames are selected for processing. This next batch of image frames is processed in a similar manner as described herein with respect to steps 112 to 116. Processing repeats until, at step 118, no more image frames remain to be processed. In a real-time video acquisition and signal processing environment, no more batches of image frames would be selectable when the video imaging device ceases further video image capture of the subject. In this embodiment, further processing stops. In another embodiment, the respiratory signal is continuously communicated to a display device for review by a medical professional. The continuous respiratory signal may be communicated to a storage device for storage and subsequent retrieval or communicated to a remote device over a network.

In yet another embodiment, the subject in the video is being monitored for the occurrence of a physiological event such as: Sudden Infant Death Syndrome (SIDS), respiratory distress, respiratory failure, sleep apnea, or pulmonary disease. In this embodiment, the respiratory signal would be continuously post-processed for the occurrence of the physiological event for which the subject is being monitored. If, an alert condition is determined to have occurred by either a visual examination of the respiratory signal or by an algorithm monitoring the respiratory signal, and alert signal or notification can be sent to a technician, nurse, medical practitioner, and the like, to alert them that there is a respiratory condition which requires their attention. The alert signal may be communicated via network and may take the form of a message or, for instance, a bell tone or a sonic alert being activated at a nurse's station. The alert signal may take the form of initiating a visible light which provides an indication such as, for instance, a blinking colored light. The alert can be a text, audio, and/or video message. Thereafter, additional actions would be taken in response to the alert signal.

It should be appreciated that the teachings hereof are intended to be used in a continuous manner for patient monitoring where the image frames of the video captured by the video imaging device are continuously processed in real-time as they are received and a respiratory signal is continuously generated as a result of processing batches of the received image frames in accordance with the methods disclosed herein. It should also be appreciated that the flow diagrams depicted herein are illustrative. One or more of the operations illustrated in the flow diagrams may be performed in a differing order. Other operations may be added, modified, enhanced, or consolidated. Variations thereof are intended to fall within the scope of the appended claims.

### Block Diagram of Video Processing System

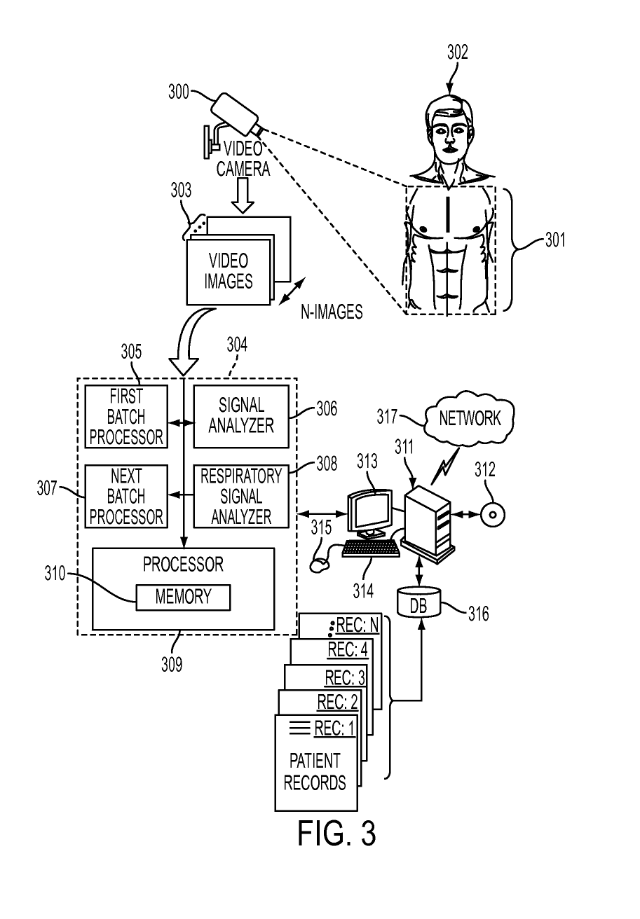

Reference is now being made to FIG. 3 which shows a block diagram of one example video processing system 300 for processing a video in accordance with the embodiment shown and described with respect to the flow diagrams of FIGS. 1-2.

In FIG. 3, video camera 300 is shown actively acquiring a streaming video of the body region 301 of the subject 302. Video images (collectively at 303) are communicated to Video Processing System 304. First Batch Processor 305 receives the video image frames 303 of the body region 301 where a time-varying signal corresponding to a respiratory function of the subject can be registered by the video camera 300. Pixels of the body region in the image frames of the received first batch of image frames are processed to obtain a time-series signal. The time-series signal is then filtered using a first band-pass filter with a default low and high cutoff frequency f_{L} and f_{H}, where f_{L} and f_{H} are at least a function of the subject's tidal breathing. The filtered time-series signal is then communicated to Signal Analyzer 306 wherein a low and high cutoff frequency f′_{L} and f′_{H} is identified, where f_{L}<f′_{L} and f′_{H}<f_{H}. The identified low and high cutoff frequency f′_{L} and f′_{H} are provided to next Next Batch Processor 307 which, in accordance with one embodiment of the present method, iteratively processes pixels in the body region of successive time-sequential batches of image frames to obtain a next sequential time-series signal associated with each next successive batch of image frames. On each successive iteration, the next sequential time-series signal is filtered using a second band-pass filter with the identified low and high cutoff frequencies f′_{L} and f′_{H}. The filtered next sequential time-series signals are filtered, respectively, to obtain a respiratory signal for the subject 302. In this embodiment, the respiratory signal is generated in real-time as the video image frames are received and processed by Respiratory Signal Analyzer 308 to determine whether a respiratory condition is occurring which requires attention. Processor 309 retrieves machine readable program instructions from Memory 310 and is provided to facilitate the functionality of any of the modules of the video processing system 304. The processor, operating alone or in conjunction with other processors and memory, may be configured to assist or otherwise facilitate the functionality of any of the processors and modules of system 404.

Video processing system 304 is shown in communication with a workstation 311. A computer case of the workstation houses various components such as a motherboard with a processor and memory, a network card, a video card, a hard drive capable of reading/writing to machine readable media 312 such as a floppy disk, optical disk, CD-ROM, DVD, magnetic tape, and the like, and other software and hardware needed to perform the functionality of a computer workstation. The workstation further includes a display device 313, such as a CRT, LCD, or touchscreen device, for displaying information, video, measurement data, computed values, medical information, results, locations, and the like. A user can view any of that information and make a selection from menu options displayed thereon. Keyboard 314 and mouse 315 effectuate a user input or selection. The workstation 311 implements a database in storage device 316 wherein patient records are stored, manipulated, and retrieved in response to a query. Such records, in various embodiments, take the form of patient medical history stored in association with information identifying the patient along with medical information. Although the database is shown as an external device, the database may be internal to the workstation mounted, for example, on a hard disk therein.

It should be appreciated that the workstation has an operating system and other specialized software configured to display alphanumeric values, menus, scroll bars, dials, slideable bars, pull-down options, selectable buttons, and the like, for entering, selecting, modifying, and accepting information needed for processing video image frames and respiratory signals in accordance with the teachings hereof. The workstation is further enabled to display the image frames comprising the video. In other embodiments, a user or technician may use the user interface of the workstation to identify areas of interest, set parameters, select image frames and/or regions of images for processing. These selections may be stored/retrieved in a storage devices 312 and 316. Default settings and initial parameters can be retrieved from any of the storage devices shown, as needed.

Although shown as a desktop computer, it should be appreciated that the workstation 311 can be a laptop, mainframe, or a special purpose computer such as an ASIC, circuit, or the like. The embodiment of the workstation of FIG. 3 is illustrative and may include other functionality known in the arts. Any of the components of the workstation 311 may be placed in communication with the video processing system 304 or any devices in communication therewith. Moreover, any of the modules and processing units of video processing system can be placed in communication with storage device 316 and/or computer media 312 and may store/retrieve therefrom data, variables, records, parameters, functions, and/or machine readable/executable program instructions, as needed to perform their intended functions. Each of the modules of the video processing system may be placed in communication with one or more remote devices over network 317. It should be appreciated that some or all of the functionality performed by any of the modules or processing units of the video processing system can be performed, in whole or in part, by the workstation placed in communication with the video imaging device 300 over network 317. The embodiment shown is illustrative and should not be viewed as limiting the scope of the appended claims strictly to that configuration. Various modules may designate one or more components which may, in turn, comprise software and/or hardware designed to perform the intended function.

The teachings hereof can be implemented in hardware or software using any known or later developed systems, structures, devices, and/or software by those skilled in the applicable art without undue experimentation from the functional description provided herein with a general knowledge of the relevant arts. One or more aspects of the methods described herein are intended to be incorporated in an article of manufacture which may be shipped, sold, leased, or otherwise provided separately either alone or as part of a product suite or a service.

It will be appreciated that the above-disclosed and other features and functions, or alternatives thereof, may be desirably combined into other different systems or applications. Presently unforeseen or unanticipated alternatives, modifications, variations, or improvements may become apparent and/or subsequently made by those skilled in this art which are also intended to be encompassed by the following claims. The teachings of any publications referenced herein are each hereby incorporated by reference in their entirety.

What is claimed is:

1. A method for processing image frames of a video of a subject for respiratory function analysis in a non-contact, remote sensing environment, the method comprising:
receiving a video comprising a plurality of time-sequential image frames of at least a body region of a subject where a time-varying signal corresponding to a respiratory function can be registered by a video camera used to capture said video;
processing pixels in said body region in a first batch of image frames to obtain a time-series signal;
filtering said time-series signal using a first band-pass filter with a low and high cutoff frequency f_{L} and f_{H}, where f_{L} and f_{H} are at least a function of said subject's tidal breathing;
analyzing said filtered time-series signal to identify a low and high cutoff frequency f′_{L} and f′_{H}, where f_{L}<f′_{L} and f′_{H}<f_{H}; and
for each next successive batch of image frames:
processing pixels in said body region in said next successive batch of image frames to obtain a next sequential time-series signal;
filtering said next sequential time-series signal using a second band-pass filter with said low and high cutoff frequencies f′_{L} and f′_{H}; and
processing said filtered next sequential time-series signal to obtain a respiratory signal for said subject.

2. The method of claim 1, wherein said video images comprise any combination of: monochrome images, color images, infrared (IR) images, multispectral images, and hyperspectral video images.

3. The method of claim 1, wherein said low and high cutoff frequencies are a function of any of: said subject's respiratory health, and said subject's age.

4. The method of claim 1, wherein a size of a batch of video frames is at least 3 breathing cycles of said subject.

5. The method of claim 1, wherein said body region is any of: an anterior thoracic region of said subject, a side view of said thoracic region, and a back region of said subject's dorsal body.

6. The method of claim 1, wherein processing pixels further comprises isolating pixels in said image frames associated with said subject's body region using any of: pixel classification, object identification, thoracic region recognition, color, texture, spatial features, spectral information, pattern recognition, and a user input.

7. The method of claim 1, further comprising, in advance of filtering, detrending said time-series signal to remove low frequency variations and non-stationary components.

8. The method of claim 1, wherein processing said filtered time-series signal to obtain said respiratory signal comprises any of:
performing a non-parametric spectral density estimation on said filtered signal;
performing a parametric spectral density estimation on said filtered signal; and
performing automatic peak detection on said filtered signal.

9. The method of claim 1, wherein said first and second band-pass filters are the same.

10. The method of claim 1, further comprising analyzing said respiratory signal to determine any of: breathing pattern, and respiration rate.

11. The method of claim 1, further comprising using said respiratory signal to determine a condition related to any of: Sudden Infant Death Syndrome, respiratory distress, respiratory failure, apnea, and pulmonary disease.

12. The method of claim 1, wherein said video is a live streaming video and said respiratory signal is generated in real-time.

13. A system for processing image frames of a video of a subject for respiratory function analysis in a non-contact, remote sensing environment, the system comprising:
a memory and storage device; and
a processor in communication with a memory and storage device, said processor executing machine readable instructions for performing:
receiving a video comprising a plurality of time-sequential image frames of at least a body region of a subject where a time-varying signal corresponding to a respiratory function can be registered by a video camera used to capture said video;
processing pixels in said body region in a first batch of image frames to obtain a time-series signal;
filtering said time-series signal using a first band-pass filter with a low and high cutoff frequency f_{L} and f_{H}, where f_{L} and f_{H} are at least a function of said subject's tidal breathing;
analyzing said filtered time-series signal to identify a low and high cutoff frequency f′_{L} and f′_{H}, where f_{L}<f′_{L} and f′_{H}<f_{H}; and
for each next successive batch of image frames:
processing pixels in said body region in said next successive batch of image frames to obtain a next sequential time-series signal;
filtering said next sequential time-series signal using a second band-pass filter with said low and high cutoff frequencies f′_{L} and f′_{H}; and
processing said filtered next sequential time-series signal to obtain a respiratory signal for said subject.

14. The system of claim 13, wherein said video images comprise any combination of: monochrome images, color images, infrared (IR) images, multispectral images, and hyperspectral video images.

15. The system of claim 13, wherein said low and high cutoff frequencies are a function of any of: said subject's respiratory health, and said subject's age.

16. The system of claim 13, wherein a size of a batch of video frames is at least 3 breathing cycles of said subject.

17. The system of claim 13, wherein said body region is any of: an anterior thoracic region of said subject, a side view of said thoracic region, and a back region of said subject's dorsal body.

18. The system of claim 13, wherein processing pixels further comprises isolating pixels in said image frames associated with said subject's body region using any of: pixel classification, object identification, thoracic region recognition, color, texture, spatial features, spectral information, pattern recognition, and a user input.

19. The system of claim 13, further comprising, in advance of filtering, detrending said time-series signal to remove low frequency variations and non-stationary components.

20. The system of claim 13, wherein processing said filtered time-series signal to obtain said respiratory signal comprises any of:
performing a non-parametric spectral density estimation on said filtered signal;
performing a parametric spectral density estimation on said filtered signal; and
performing automatic peak detection on said filtered signal.

21. The system of claim 13, wherein said first and second band-pass filters are the same.

22. The system of claim 13, further comprising analyzing said respiratory signal to determine any of: breathing pattern, and respiration rate.

23. The system of claim 13, further comprising using said respiratory signal to determine a condition related to any of: Sudden Infant Death Syndrome, respiratory distress, respiratory failure, apnea, and pulmonary disease.

24. The system of claim 13, wherein said video is a live streaming video and said respiratory signal is generated in real-time.
